(19) United States
(12) Patent Application Publication
(10) Pub. No.: US 2024/0100146 A1
(43) Pub. Date: Mar. 28, 2024

(54) Neuraminidase-Inhibited Influenza Virus

(71) Applicant: VaxDome Inc., Fort Worth, TX (US)

(72) Inventor: De-chu Christopher Tang, Fort Worth, TX (US)

(21) Appl. No.: 18/470,999

(22) Filed: Sep. 20, 2023

Related U.S. Application Data
(60) Provisional application No. 63/408,387, filed on Sep. 20, 2022.

(51) Int. Cl. A61K 39/145; A61P 31/14; A61P 31/16

(52) U.S. Cl. CPC A61K 39/145; A61P 31/14; A61P 31/16; A61K 2039/5254

(57) ABSTRACT

A composition, method of making, and method of using an active agent against a myriad of known as well as unknown pathogens comprising: binding a live influenza virus with a neuraminidase inhibitor in vitro; and administering the active agent with or without elimination of unbound neuraminidase inhibitors by intranasal administration or oral inhalation into patients. The active agent confers rapid and broad protection to a patient performing at least one of: i) elicit an innate immune response against known or unknown pathogens; ii) mitigate lymphopenia in general; iii) enable natural infection to activate adaptive immunity by allowing pathogens to harmlessly linger in an infected patient for a limited amount of time; and/or iv) eliciting protective immunity against influenza virus as a rapid-response influenza vaccine by making a clinically-isolated influenza virus immediately benign with a neuraminidase inhibitor in vitro without the time-consuming requirement to generate conventional influenza vaccines.

### CROSS-REFERENCE TO RELATED APPLICATIONS

This application claims priority to U.S. Provisional Application Ser. No. 63/408,387, filed Sep. 20, 2022, the entire contents of which are incorporated herein by reference.

### TECHNICAL FIELD OF THE INVENTION

The present invention relates in general to the field of anti-pathogen innate immunity, pathogen/mutation-agnostic vaccine potentiator, mitigation of lymphopenia, mitigation of virus-induced death of T cells, recruitment of T cells into the lung, amplification of interferon responses, rapid-response mutation-agnostic influenza vaccine, and more particularly, to a novel composition and method for making an active agent capable of elicitation of pathogen/mutation-agnostic innate immunity, or allowing a pathogen to linger harmlessly in an infected patient for a limited amount of time as a post-infection vaccine, or generation of a new influenza vaccine rapidly by mixing a villain influenza virus with a neuraminidase inhibitor as a rapid-response mutation-agnostic influenza vaccine.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with antivirals and vaccines, respectively.

Infection of the respiratory tract by airborne viruses is frequent and ubiquitous. The numerous variants of influenza virus and severe acute respiratory syndrome coronavirus (SARS-CoV-2) have been ravaging public health worldwide with no end in sight, and there is global consensus that contemporary countermeasures against airborne viruses are hugely insufficient.

Vaccine's disease-fighting power has defeated many pathogens as a medical bonanza credited with global reduction of mortality and morbidity; however, vaccination has been less than adequate against airborne RNA viruses including influenza virus (IFV) and the severe acute respiratory syndrome coronavirus 2 (SARS-CoV-2). Even though the seasonal trivalent inactivated influenza vaccine (TIV) is deemed a mainstay in mitigating influenza, repeat influenza vaccinations over multiple seasons have shown that repeat inoculations of kindred IFV-derived antigens would enfeeble a vaccine's disease-fighting power against an IFV (Sanyal et al., 2019). In addition, elicitation of protective immunity by vaccination is too slow to protect people who are at an immediate risk and TIV generally fails to elicit heterosubtypic immunity (Tang et al., 2009). Similar to IFV with an RNA genome that mutates relentlessly, the SARS-CoV-2 also tends to evolve into many unpredictable variants that emerge to haunt the public. Although mass-immunizations of human populations with a variety of licensed COVID-19 vaccines have effectively reduced the rates of COVID-19-mediated death and severe disease (Telenti et al., 2021), COVID-19 vaccines' crescendo is vitiated every time escape mutants emerge (Bernal et al., 2021). Undesirably, the effectiveness of the licensed COVID-19 vaccines wanes in merely a few months post-vaccination (Andrews et al., 2022). Moreover, the Achilles heel of intramuscular vaccination, as commonly practiced, is its incompetence to confer protection against airborne infection in the upper respiratory tract and its failure to block virus shedding (Lund and Randall, 2021; van Doremalen et al., 2021). Compelling evidence indicates that the licensed intramuscular vaccines, as they are, would be part of a solution to mitigate respiratory diseases, but have failed to fully address future airborne virus-induced pandemics or seasonal outbreaks.

To fortify the arsenal against airborne infections, it is crucial to foster the development of multipronged approaches against respiratory viruses. Therapy options capable of broadly reducing the severity of airborne infections are of paramount utility. The M2 ion channel blockers, amantadine and rimantadine, and the neuraminidase inhibitors, oseltamivir (Tamiflu) and zanamivir (Relenza) have proven effective in treating IFV infections; however, these influenza drugs tend to generate drug-resistant IFV strains over time (Poland et al., 2009). Although a number of drug candidates including dexamethasone (Tomazini et al., 2020), remdesivir (Parums, 2022), anti-spike monoclonal antibodies (Wang et al., 2021) have been evaluated in treating COVID-19 patients, their efficacies are limited in mitigating this devastating disease. Ominously, escape mutants of SARS-CoV-2 that are resistant to a subset of anti-spike monoclonal antibodies are in circulation (Wang et al., 2021), and early treatment of COVID-19 patients with dexamethasone is linked to increased mortality (Servick et al., 2021; Swaminathan et al., 2022). The oral antiviral prodrug molnupiravir can mildly reduce the risk of death in COVID-19 patients (Bernal et al., 2022); whereas, paxlovid appears to be a more effective therapeutic option to prevent progression of COVID-19 to severe disease (Kozlov, 2022). However, drug resistance is a debacle-in-waiting for “monotherapies” such as molnupiravir and paxlovid that each targets only one part of the virus. Sinister evidence has shown that paxlovid-resistant SARS-CoV-2 mutants could be generated after propagation of SARS-CoV-2 in permissive cells under selective pressure of paxlovid (Service, 2022; Sidik, 2022). Furthermore, SARS-CoV-2 tends to rebound in a subset of paxlovid-treated patients post-therapy (Rubin, 2022; Service, 2022). There is thus an urgent need to develop more powerful antivirals capable of arresting SARS-CoV-2 and other viruses from different angles. It would be hugely beneficial to public health if a pathogen/mutation-agnostic antiviral capable of countering a myriad of airborne viruses without the potential to induce drug resistance should be developed. The more antivirals are in the arsenal, the more resilient public health could be.

As such, a need remains for novel anti-viral agents and strategies that can be used to rapidly address future airborne virus-induced pandemics or seasonal outbreaks.

### SUMMARY OF THE INVENTION

As embodied and broadly described herein, an aspect of the present disclosure relates to a method of treating a patient with an active agent capable of generating a pathogen/mutation-agnostic innate immune response, acting as an adaptive immune response potentiator, or as a rapid-response mutation-agnostic influenza vaccine, the method comprising: mixing a live influenza virus with a neuraminidase inhibitor in vitro to form the active agent with or without eliminating the unbound neuraminidase inhibitor; and administering the active agent to the patient, wherein the active agent performs at least one of: eliciting a pathogen/mutation-agnostic innate immune response in the patient, potentiating an adaptive immune response against a pathogen, or generating an immune response against influenza. In one aspect, the influenza virus is a natural influenza virus, a bioengineered influenza virus, or a cold-adapted influenza virus. In another aspect, the active agent is administered with or without eliminating the neuraminidase inhibitor. In another aspect, the neuraminidase inhibitor is zanamivir, oseltamivir carboxylate, laninamivir, peramivir, or any other neuraminidase inhibitor. In another aspect, the pathogen is a virus, a bacterium, or a fungus. In another aspect, the active agent is inoculated into animals or human subjects by intranasal administration or oral inhalation. In another aspect, the method further comprises administering the active agent to mitigate virus-induced lymphopenia or thymic atrophy or both. In another aspect, the method further comprises administering the active agent to mitigate virus-induced death of T cells. In another aspect, the method further comprises administering the active agent to recruit T cells into the lung. In another aspect, the method further comprises administering the active agent to trigger an antiviral response by amplifying virus-induced interferon production. In another aspect, the active agent triggers an innate immune response, an adaptive immune response, or both. In another aspect, the virus is at least one of: an influenza virus, a coronavirus, a respiratory syncytial virus, a rhinovirus, or a measles virus. In another aspect, the bacterium is Bacillus, Clostridium, Mycobacterium, Staphylococcus, Streptococcus, Pseudomonas, Klebsiella, Haemophilus, or Mycoplasma. In another aspect, the fungus is Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof. In another aspect, the method further comprises administering the active agent to mitigate lymphopenia induced by radiation, senescence, inflammation, infection, or combinations thereof.

As embodied and broadly described herein, an aspect of the present disclosure relates to an active agent comprising a live influenza virus treated with a neuraminidase inhibitor in vitro formulated for nasal or oral administration.

As embodied and broadly described herein, an aspect of the present disclosure relates to a method of treating a patient suspected of having an infectious disease or having a high risk of contracting an infectious agent comprising: identifying the patient in need of treatment for a pathogen; and providing the patient with an effective amount of an active agent comprising a live influenza virus treated with a neuraminidase inhibitor in vitro, wherein the active agent triggers a protective immune response against the pathogen, wherein the immune response is selected from at least one of: an innate immune response, an adaptive immune response, or both.

As embodied and broadly described herein, an aspect of the present disclosure relates to a method of potentiating an adaptive immune response to a pathogen comprising: providing the patient with an effective amount of an active agent comprising a live influenza virus treated with a neuraminidase inhibitor in vitro which allows a pathogen to harmlessly linger in an infected patient for a limited amount of time as a post-infection natural vaccine capable of triggering a protective immune response against the pathogen, wherein the immune response is an adaptive immune response. In one aspect, the pathogen is a virus, a bacterium, or a fungus. In another aspect, the virus is at least one of: an influenza virus, a coronavirus, a respiratory syncytial virus, a rhinovirus, or a measles virus. In another aspect, the bacterium is Bacillus, Clostridium, Mycobacterium, Staphylococcus, Streptococcus, Pseudomonas, Klebsiella, Haemophilus, or Mycoplasma. In another aspect, the fungus is Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof.

As embodied and broadly described herein, an aspect of the present disclosure relates to a method of immunizing a patient against influenza by nasal spray or oral inhalation of a live influenza virus treated with a neuraminidase inhibitor in vitro. In one aspect, the live influenza virus is a clinically-isolated influenza virus. In another aspect, the live influenza virus is a bioengineered non-cold-adapted influenza virus. In another aspect, the live influenza virus is a cold-adapted live attenuated influenza virus (LAIV). In another aspect, the live influenza virus is a bioengineered LAIV.

### BRIEF DESCRIPTION OF THE DRAWINGS

For a more complete understanding of the features and advantages of the present invention, reference is now made to the detailed description of the invention along with the accompanying figures and in which:

FIGS. 1A to 1G show that strong anti-IFV and anti-CoV protective immune responses are elicited in mice by intranasal (i.n.) instillation of zIFV-PR8 particles. FIG. 1A shows that 100% (9/9) of A/J mice challenged by intranasal (i.n.) instillation of PR8 on Day 0 succumbed to influenza within 7 days post-challenge whereas 100% (10/10) of mice challenged by i.n. instillation of zIFV-PR8 on Day 0 survived. FIG. 1B shows that i.n. instillation of zIFV-PR8 into mice conferred protection of mice (89%; 8/9) 8 days later against a lethal challenge with MHV1 whereas i.n. instillation of zanamivir without PR8 conferred no protection (0%; 0/8). FIG. 1C shows that i.n. instillation of zIFV-PR8 conferred transient protection of mice against a lethal challenge with MHV1. FIG. 1D shows that i.n. instillation of zIFV-PR8 into mice conferred long-term protection of mice against a lethal challenge with PR8 as a rapid-response influenza vaccine, after zIFV-PR8-induced protective innate immunity declined away (FIG. 1C). FIG. 1E shows that zIFV-PR8 could convert the virulent MHV1 into a coronavirus vaccine as a vaccine potentiator. MHV1 transiently harnessed by zIFV-PR8-induced protective innate immunity in infected mice conferred long-term protection of mice against repeat infections by MHV1. FIG. 1F shows that i.n. instillation of a second dose of zIFV-PR8 into zIFV-PR8-immunized mice still conferred partial protection of mice against a lethal challenge with MHV1, whereas i.n. instillation of virulent PR8 into zIFV-PR8-immunized mice prior to MHV1 challenge exacerbated WW1's lethal effect, even though PR8 itself did not kill mice due to zIFV-PR8-mediated protection against PR8. FIG. 1G shows that zIFV-PR8 replicates to a low titer within the lung for at least 8 days post-i.n. administration.

FIGS. 2A to 2C show the zPR8-mediated amplification of CoV-induced interferon (IFN) responses within lungs in A/J mice. FIG. 2A shows that i.n. instillation of zIFV-PR8 did not appreciably induce production of IFN-α within the lung; however, the level of IFN-α in MHV1-infected and zIFV-PR8-immunized animals tended to be higher than that in MHV1-infected animals without zIFV-PR8 immunization, as well as that in zIFV-PR8-immunized animals without MHV1 infection, perhaps owing to amplification of MHV1-induced IFN-α response by zIFV-PR8 which was incompetent in triggering an IFN-α response by itself. FIG. 2B shows that i.n. instillation of zIFV-PR8 did not appreciably induce production of IFN-β within the lung; however, the level of IFN-β in MHV1-infected and zIFV-PR8-immunized animals tended to be higher than that in MHV1-infected animals without zIFV-PR8 immunization, as well as that in zIFV-PR8-immunized animals without MHV1 infection, even though the differences did not reach statistical significance. FIG. 2C shows that i.n. instillation of zIFV-PR8 did not appreciably induce production of IFN-γ within the lung; however, the level of IFN-γ in MHV1-infected and zIFV-PR8-immunized animals tended to be higher than that in MHV1-infected animals without zIFV-PR8 immunization, as well as that in zIFV-PR8-immunized animals without MHV1 infection, even though the differences did not reach statistical significance. FIG. 2D shows that the mouse coronavirus MHV1 titers in lungs could be suppressed by prior exposure to zIFV-PR8.

FIGS. 3A to 3D show the lung histopathology induced by i.n. instillation of zIFV and/or MHV1. (FIG. 3A) Lung resected from an untreated normal A/J mouse. (FIG. 3B) Lung resected from an A/J mouse 12 days after i.n. instillation of zIFV-PR8 (800 TCID₅₀ of PR8 mixed with 500 μg of zanamivir). (FIG. 3C) Lung resected from an A/J mouse 4 days after i.n. instillation of MHV1 (2×10⁴ TCID₅₀). (FIG. 3D) Lung resected from an A/J mouse with zIFV-PR8 inoculated by i.n. instillation on Day 0, followed by i.n. instillation of MHV1 on Day 12, with the lung resected on Day 16.

FIGS. 4A to 4L show the tug of war between zIFV-PR8 and MHV1 in controlling the numbers of CD4⁺ and CD8⁺ T cells in the lung. (FIG. 4A) CD4⁺ T cells within alveolar spaces in a lung resected from an A/J mouse 12 days after i.n. instillation of zIFV-PR8 (800 TCID₅₀ of PR8 mixed with 500 μg of zanamivir). (FIG. 4B) CD4⁺ T cells within foci in the same lung as shown in (FIG. 4A). (FIG. 4C) CD4⁺ T cells within alveolar spaces in a lung resected from an A/J mouse 4 days after i.n. instillation of MHV1 (2×10⁴ TCID₅₀). (FIG. 4D) CD4⁺ T cells within inflamed region in the same lung as shown in (FIG. 4C). (FIG. 4E) CD4⁺ T cells within alveolar spaces in a lung resected from an A/J mouse with zIFV-PR8 administered by i.n. instillation on Day 0, followed by i.n. instillation of MHV1 on Day 12, with the lung resected for IHC analysis on Day 16. (FIG. 4F) CD4⁺ T cells within foci in the same lung as shown in (FIG. 4E). (FIG. 4G) CD8⁺ T cells within alveolar spaces in the same lung as shown in (FIG. 4A). (FIG. 4H) CD8⁺ T cells within foci in the same lung as shown in (FIG. 4A). (FIG. 4I) CD8⁺ T cells within alveolar spaces in the same lung as shown in (FIG. 4C). (FIG. 4J) CD8⁺ T cells within inflamed region in the same lung as shown in (FIG. 4C). (FIG. 4K) CD8⁺ T cells within alveolar spaces in the same lung as shown in (FIG. 4E). (FIG. 4L) CD8⁺ T cells within foci in the same lung as shown in (FIG. 4E). FIG. 4M shows that i.n. instillation of zIFV-PR8 into A/J mice recruited a large number of CD3⁺ T cells (brown cells revealed during immunohistochemical analysis) into the lung. These T cells were found in both the alveolar spaces and foci. FIG. 4N shows that very few CD3⁺ T cells were found in lungs after i.n. instillation of MHV1 into A/J mice. In addition, no foci were found either. FIG. 4O shows that mice immunized by i.n. instillation of zIFV-PR8 followed by i.n. instillation of MHV1 12 days later, CD3⁺ T cells were still abundant within foci; however, these T cells were largely absent in alveolar spaces. FIG. 4P shows that naïve A/J mice had a low level of CD3⁺ T cells within lungs prior to immunization with zIFV-PR8.

FIGS. 5A to 5F summarize the tug of war between zIFV-PR8-induced lymphocytosis and MHV1-induced lymphopenia in A/J mice. (FIG. 5A) Body weight loss post-MHV1 challenge. Post-MHV1 challenge body weights were presented as mean % body weight by taking the body weight of individual mice on Day 0 as 100%. Naïve, control A/J mice without treatment; zPR8-12d, zIFV-PR8 (800 TCID₅₀ of PR8 mixed with 500 μg of zanamivir) were administered into each mouse by i.n. instillation, with mice euthanized 12 days later; MHV1-4d, MHV1 (2×10⁴ TCID₅₀) were administered into each mouse by i.n. instillation, with mice euthanized 4 days later; zPR8-8d−MHV1-4d, zIFV-PR8 were administered into each mouse by i.n. instillation on Day 0, followed by i.n. instillation of MHV1 on Day 8 (shown as Day 0 in FIG. 5A), with mice euthanized on Day 12 (shown as Day 4 in FIG. 5A). Numbers in parentheses represent the number of animals in each group. Mice in the MHV1-4d group significantly lost body weight over time when compared to their counterparts in other groups (unpaired t-test). Statistical significance was set at P<0.05. (FIG. 5B) MHV1-induced thymic atrophy. Normal, thymus glands resected from naïve A/J mice; zPR8-12, thymus glands from mice in the zPR8-12d group; MHV1-4d, thymus glands from the MHV1-4d group; zPR8-8d−MHV1-4d, thymus glands from the zPR8-8d−MHV1-4d group. (FIG. 5C) Representative flowcytometric gating of thymocytes in each group of mice. (FIG. 5D) Summarized data of total thymocyte number (topmost), thymocyte number/mouse body weight (second top), percentage of thymocyte subsets (second bottom), and number of thymocyte subsets (bottommost) in each group of mice. (FIG. 5E) Representative flowcytometric gating of lymphocytes in the peripheral blood in each group of mice. (FIG. 5F) Summarized data of percentage of lymphocytes in the peripheral blood in each group of mice. Naïve, naïve A/J mice without treatment; V20, i.n. instillation of 30 μl of V20 buffer (virus storage buffer) into each mouse 4 days prior to euthanasia; MHV1, the MHV1-4d group; zPR8+MHV1, the zPR8-8d−MHV1-4d group, zPR8, the zPR8-12d group.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The recent COVID-19 pandemic accentuated the urgency to develop broad-spectrum antivirals without recourse to specific viral antigens against a myriad of airborne viruses. It is demonstrated herein that intranasal administration of zanamivir-crippled influenza virus (zPR8) particles conferred rapid protection of mice against lethal challenges by different influenza virus strains or a coronavirus strain in an adaptive immunity-independent manner. The pathogen-agnostic protective effects triggered by a single dose of zPR8 persisted for several weeks, and was extendable by a booster instillation. zPR8 itself failed to induce appreciable production of interferons-α, -β, and -γ within the lung post-administration; however, zPR8 conceivably amplified coronavirus-induced interferon responses. Intranasal challenge of mice with coronavirus rapidly induced lymphopenia associated with thymic atrophy whereas zPR8 could counter coronavirus-induced lymphopenia by inducing lymphocytosis and extricate infected animals from coronavirus-induced lethal effects. Eradication of live coronaviruses was not a prerequisite for zPR8 to trigger a pathogen-agnostic protective effect. The lingering coronaviruses in a zPR8-immunized animal conferred no harm to the host and activated adaptive immunity as a natural vaccine against repeat infections. This transition by inhibition of viral neuraminidase with zanamivir in vitro followed by administration of neuraminidase-inhibited IFV particles into patients generates broad-spectrum antiviral and infection-dependent vaccine potentiator and an influenza vaccine capable of mobilizing the immune repertoire toward beneficial protection against known as well as unknown viruses in a simple, effective, economical, and safe manner, with neither the potential to induce drug resistance nor the frantic requirement to develop belated vaccines when another unknown pathogen strikes.

It was recently found that, surprisingly, a neuraminidase-inhibited IFV (zIFV-PR8) can be easily generated by mixing the mouse-adapted IFV A/Puerto Rico/8/34 (PR8) with a neuraminidase inhibitor, e.g., zanamivir, in vitro. It was found that zanamivir is able to quickly transmogrify the pathogenic IFV into a beneficial pathogen-agnostic antiviral that confers rapid protection of mice against either related or unrelated respiratory viruses in an adaptive immunity-independent manner. Titration of live CoV within the lung after infection of zIFV-PR8-immunized mice with CoV showed that zIFV-PR8 conferred superb protection of mice against CoV-induced death in the presence of CoV. Inexplicably, zIFV-PR8 amplified CoV-induced production of IFN-α, -β, and -γ. zIFV-PR8 also mitigated CoV-induced lymphopenia and thymic atrophy by inducing lymphocytosis. In addition, a virulent CoV could linger harmlessly at a low level for many days post-infection in a zIFV-PR8-immunized lung as a natural vaccine against repeat infections. The reason why PR8 kills infected animals; whereas zIFV-PR8 saves lives against virus-induced lethal effects, could be attributed to zanamivir-mediated detention of replicating IFV particles within a limited number of infected cells by disabling neuraminidase which is required for release of progeny viruses to wreak havoc on health (Gubareva et al., 2000); thus broadcasting an “infection signal” without a full-blown conflagration for the immune system to take adequate time during activation of effective reactions to ensnare the villain viruses. Overall, zIFV represents a broad-spectrum antiviral plus a vaccine potentiator plus an influenza vaccine in one package that belongs to a class of its own capable of tipping the scales of immunity toward beneficial protection of hosts against virulent viruses without attacking the villain viruses directly.

zIFV as a pathogen/mutation-agnostic antiviral/vaccine potentiator/influenza vaccine combo. Development of a pathogen/mutation-agnostic antiviral/vaccine potentiator/influenza vaccine combo capable of arresting a myriad of airborne viruses including SARS-CoV-2 would add a powerful tool to the public health arsenal against future pandemics as well as seasonal outbreaks associated with unknown viruses or new viral strains with unknown mutations.

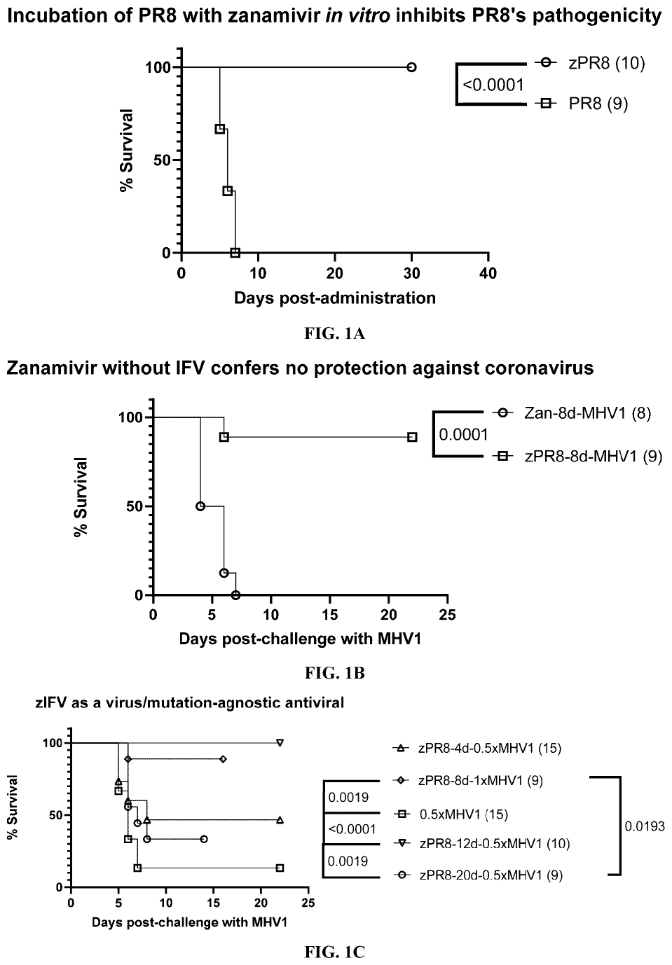

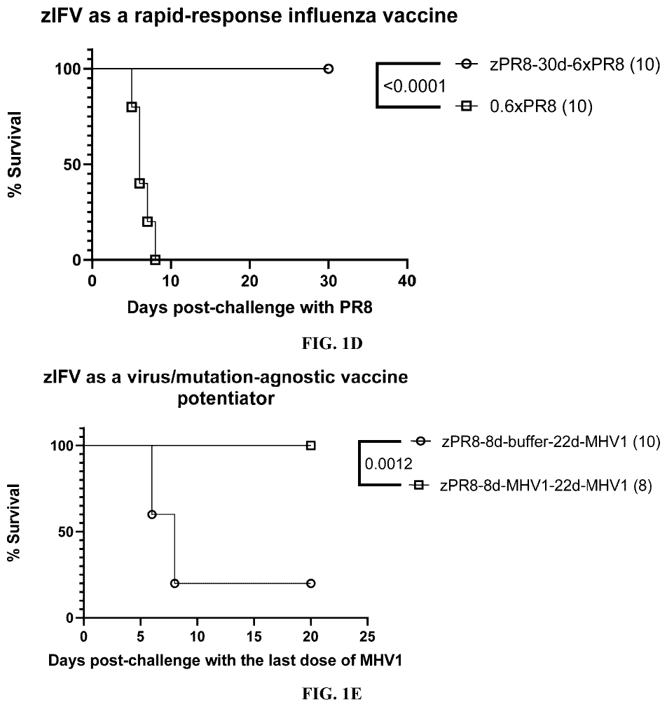

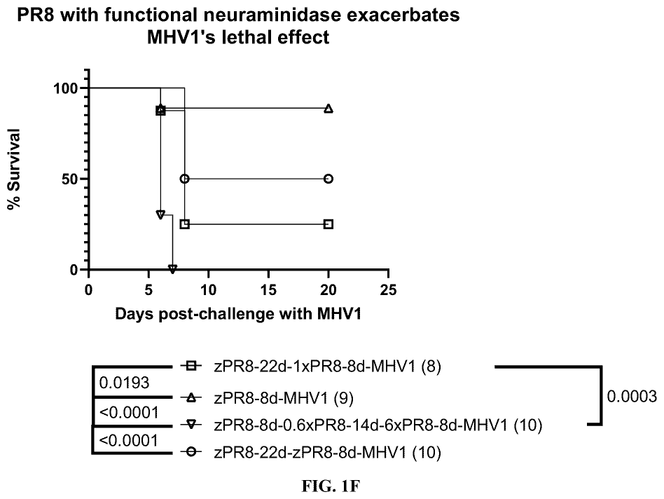

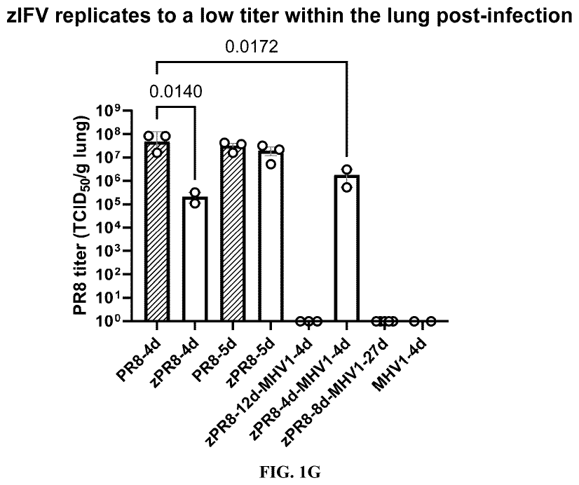

FIG. 1A shows that 100% (9/9) of A/J mice challenged by intranasal (i.n.) instillation of PR8 on Day 0 succumbed to influenza within 7 days post-challenge whereas 100% (10/10) of mice challenged by i.n. instillation of zIFV-PR8 on Day 0 survived. The difference owing to zanamivir reached statistical significance when the two groups were compared (P<0.0001). % survival was determined by taking the number of mice on Day 0 as 100%. Numbers in parentheses represent the number of animals in each group. Results suggest that PR8's pathogenicity could be inhibited by incubation of PR8 with the neuraminidase inhibitor zanamivir in vitro. zPR8 group, the mouse-adapted influenza virus (IFV) A/Puerto Rico/8/34 (PR8) was incubated with zanamivir at a ratio of 800 50% tissue culture infectious dose (TCID₅₀) PR8/500 μg zanamivir dissolved in phosphate buffer solution (PBS) for 1-3 hours in a volume of 0.05 ml which was the dose administered into each mouse in this group; PR8 group, 800 TCID₅₀ of PR8 in a volume of 0.05 ml PBS per mouse. Statistical analysis was performed using GraphPad Prism software. Log-rank tests were performed for comparing Kaplan-Meier survival curves. Statistical significance was set at P<0.05.

FIG. 1B shows that i.n. instillation of zIFV-PR8 into mice conferred protection of mice (89%; 8/9) 8 days later against a lethal challenge with MHV1 whereas i.n. instillation of zanamivir without PR8 conferred no protection (0%; 0/8). The protection afforded by zIFV-PR8 reached statistical significance (P=0.0001) when compared to that of zanamivir alone without PR8. Zan-8d−MHV1 group, i.n. instillation of 500 μg zanamivir dissolved in 0.05 ml PBS into each mouse followed by MHV1 challenge 8 days later; zPR8-8d−MHV1 group, i.n. instillation of zIFV-PR8 into each mouse followed by MHV1 challenge 8 days later; mice were challenged by i.n. instillation of 1.6×10⁴ TCID₅₀ of MHV1 in a volume of 0.03 ml per mouse. Other details were described in FIG 1A legend.

FIG. 1C shows that i.n. instillation of zIFV-PR8 conferred transient protection of mice against a lethal challenge with MHV1. Mice were partially protected (47%; 7/15) against MHV1 4 days post-zIFV-PR8 immunization; and survival rate declined to 33% (3/9) 20 days post-zIFV-PR8 immunization. However, 100% (15/15) of mice were protected when mice were challenged with MHV1 12 days post-zIFV-PR8 immunization and survival rate of 89% (8/9) was achieved 8 days post-zIFV-PR8 immunization. Mice challenged with MHV1 without zIFV-PR8 immunization had a survival rate of 13% (2/15). zPR8-4d-0.5xMHV1 group, mice challenged with 8×10³ TCID₅₀ of MHV1 4 days post-zIFV-PR8 immunization; zPR8-8d-1xMHV1 group, mice challenged with 1.6×10⁴ TCID₅₀ of MHV1 8 days post-zIFV-PR8 immunization; 0.5xMHV1 group, mice challenged with 8×10³ TCID₅₀ of MHV1; zPR8-12d-0.5xMHV1 group, mice challenged with 8×10³ TCID₅₀ of MHV1 12 days post-zIFV-PR8 administration; zPR8-20d-0.5xMHV1 group, mice challenged with 8×10³ TCID₅₀ of MHV1 20 days post-zIFV-PR8 immunization. Other details were described in FIG. 1A legend.

FIG. 1D shows that i.n. instillation of zIFV-PR8 into mice conferred long-term protection of mice against a lethal challenge with PR8 as a rapid-response influenza vaccine, after zIFV-PR8-induced protective innate immunity declined away (FIG. 1C). The protection afforded by zIFV-PR8 (100%; 10/10) reached statistical significance (P<0.0001) when compared to that of the control group (0%; 0/10). zPR8-30d-6xPR8 group, mice challenged with 4,800 TCID₅₀ of PR8 30 days post-zIFV-PR8 immunization; 0.6×PR8 group, mice challenged with 480 TCID₅₀ of PR8 without zIFV-PR8 protection. Other details were described in FIG. 1A legend.

FIG. 1E shows that zIFV-PR8 could convert the virulent MHV1 into a coronavirus vaccine as a vaccine potentiator. MHV1 transiently harnessed by zIFV-PR8-induced protective innate immunity in infected mice conferred long-term protection of mice against repeat infections by MHV1. The protection afforded by the first dose of MHV1 (100%; 8/8) administered 8 days post-zIFV-PR8 immunization reached statistical significance (P=0.0012) when compared to that of the buffer control group (20%; 2/10). zPR8-8d-buffer-22d−MHV1 group, V20 buffer (virus-storage buffer containing 10 mM Tris, pH7.4; 10 mM Histidine; 5% (w/v) sucrose; 75 mM NaCl; 1 mM MgCl₂; 0.1 mM EDTA) was i.n. instilled into each mouse in this group 8 days post-zIFV-PR8 immunization followed by i.n. instillation of 1.6×10⁴ TCID₅₀ of MHV1 22 days after buffer administration; zPR8-8d-MHV1-22d−MHV1 group, 1.6×10⁴ TCID₅₀ of MHV1 was i.n. instilled into each mouse in this group 8 days post-zIFV-PR8 immunization followed by a second dose of MHV1 22 days after the first dose. Other details were described in FIG. 1A legend.

FIG. 1F shows that i.n. instillation of a second dose of zIFV-PR8 into zIFV-PR8-immunized mice still conferred partial protection of mice against a lethal challenge with MHV1, whereas i.n. instillation of virulent PR8 into zIFV-PR8-immunized mice prior to MHV1 challenge exacerbated MHV1's lethal effect, even though PR8 itself did not kill mice due to zIFV-PR8-mediated protection against PR8. zPR8-22d-1xPR8-8d−MHV1 group, mice challenged by i.n. instillation of 800 TCID₅₀ of PR8 22 days post-zIFV-PR8 immunization followed by i.n. instillation of 1.6×10⁴ TCID₅₀ of MHV1 8 days after PR8 challenge; zPR8-8d−MHV1 group, mice challenged by i.n. instillation of 1.6×10⁴ TCID₅₀ of MHV1 8 days post-zIFV-PR8 immunization; zPR8-8d-0.6xPR8-14d-6xPR8-8d−MHV1 group, mice over-challenged with PR8 by i.n. instillation of 480 TCID₅₀ and 4,800 TCID₅₀ of PR8 8 and 22 days post-zIFV-PR8 immunization, respectively, followed by challenge with 1.6×10⁴ TCID₅₀ of MHV1 8 days after the second dose of PR8; zPR8-22d-zPR8-8d−MHV1 group, zIFV-PR8-immunized mice were immunized again with zIFV-PR8 22 days later followed by MHV1 challenge 8 days after the second dose of zIFV-PR8; number, P value when the difference between two specific groups reached statistical significance. Other details were described in FIG. 1A legend.

FIG. 1G shows that zIFV-PR8 replicates to a low titer within the lung for at least 8 days post-i.n. administration. PR8-4d group, PR8 titer (TCID₅₀ per gram of lung tissue) 4 days post-i.n. instillation of PR8 (800 TCID₅₀); zPR8-4d group, PR8 titer 4 days post-i.n. instillation of zIFV-PR8 (800 TCID₅₀ of PR8 mixed with 500 μg of zanamivir); PR8-5d group, PR8 titer 5 days post-PR8 administration; zPR8-5d group, PR8 titer 5 days post-zIFV-PR8 administration; zPR8-12d−MHV1-4d group, PR8 titer 16 days after zPR8 administration and 4 days after MHV1 administration; zPR8-4d−MHV1-4d group, PR8 titer 8 days after zPR8 administration and 4 days after MHV1 administration; zPR8-8d−MHV1-27d group, PR8 titer 35 days after zPR8 administration and 27 days after MHV1 administration; MHV1-4d group, PR8 titer 4 days post-MHV1 administration. Circle, PR8 titer (TCID₅₀ per gram of lung tissue) from each animal; column, geometric mean; red bar, geometric SD. Statistical analysis was performed using GraphPad Prism software; one-way ANOVA with Tukey's multiple comparison post-tests were performed for comparing viral titers in lungs; statistical significance was set at P<0.05. Other details were described in FIG. 1A legend.

Thus, the present invention overcomes the shortcomings associated with the contemporary influenza vaccines TIV and LAIV (cold-adapted live attenuated influenza virus vaccine; e.g., FluMist), such as, the long timelines required for updating TIV and LAIV influenza vaccines when new IFV strains emerge, TIV's association with systemic inflammation, TIV's incompetence in blocking viral infections and virus shedding in the upper respiratory tract, etc. These results show that nasal spray of the zIFV or another NAI-IFV (an influenza virus with its neuraminidase bound to a neuraminidase inhibitor) of the present invention into patients may be more potent than LAIV in eliciting protective immune responses against IFV due to replicating IFV genomes locked up in a small number of infected cells; particularly when the IFV in NAI-IFV is non-cold-adapted. By way of explanation and in no way a limitation of the present invention, the zIFV within a small number of infected cells may broadcast a stronger “infection signal” that activates the immune system than a larger number of IFV particles that are pervasive. In addition, cold-adapted LAIV only replicates for limited cycles along the superficial layer of mucosal barrier in the respiratory tract where the temperature is lower. By contrast, the zIFV of the present invention can replicate in deeper layers at physiological temperature.

As used herein, the term “potentiator” refers to the use of the zIFV to accentuate or potentiate an immune response. As shown herein, the zIFV potentiates an innate immune response, as shown by amplification of CoV-induced production of interferons. However, the zIFV is also able to potentiate an adaptive immune response, as shown by acting as a potentiator that increases the immune response to a pre-existing zIFV-harnessed viral infection. Thus, the zIFV is able to enhance an immune response in both a pathogen-agnostic, but also a pathogen specific immune response.

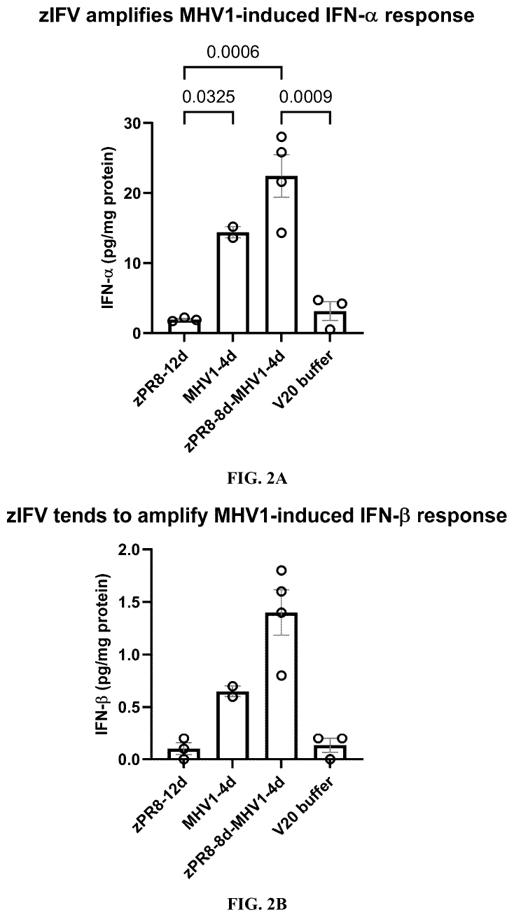

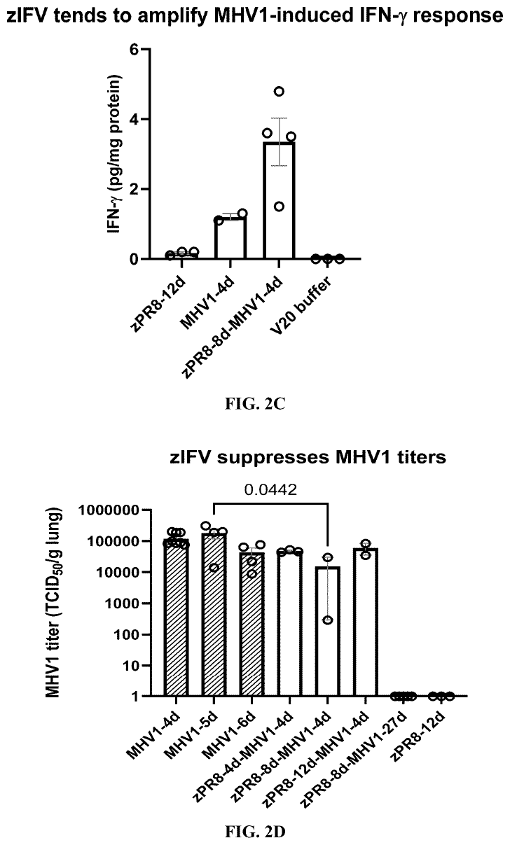

FIG. 2A shows that i.n. instillation of zIFV-PR8 did not appreciably induce production of interferon (IFN)-α within the lung; however, the level of IFN-α in MHV1-infected and zIFV-PR8-immunized animals tended to be higher than that in MHV1-infected animals without zIFV-PR8 immunization, as well as that in zIFV-PR8-immunized animals without MHV1 infection, perhaps owing to amplification of MHV1-induced IFN-α response by zIFV-PR8 which was incompetent in triggering an IFN-α response by itself. zPR8-12d group, level of IFN-α (pg/mg soluble protein) within lungs harvested 12 days post-zIFV-PR8 immunization; MHV1-4d group, level of IFN-α within lungs harvested 4 days post-MHV1 challenge; zPR8-8d−MHV1-4d group, level of IFN-α within lungs harvested 12 days post-zIFV-PR8 immunization and 4 days post-MHV1 challenge; V20 buffer group, level of IFN-α within lungs harvested 4 days after i.n. instillation of 0.03 ml of V20 buffer (virus storage buffer). Part of mouse lung was resected at an indicated time point and frozen in 1 ml of extraction buffer [PBS containing complete Mini Protease Inhibitor Cocktail (11836153001; Roche) and Triton X-100; one tablet of protease inhibitor was dissolved in 10 ml of PBS followed by adding 0.1 ml of 10% Triton X-100] at −80° C. Frozen lung tissue was subsequently thawed and homogenized in a glass tissue grinder with supernatant kept at −80° C. until assay for the level of interferons. MSD U-PLEX Interferon Combo 1 (Meso Scale Diagnostics) was utilized to analyze mouse α-, β-, and γ-interferons in duplicate lung samples according to manufacturer's protocol. 50 μL of diluted lung samples were incubated with capture antibodies, washed, incubated with detection antibodies. Interferons were recorded on a MESO QuickPlex SQ 120 reader (Meso Scale Diagnostics) and quantitated via comparison to a standard curve. DISCOVERY WORKBENCH software was used for data collection and analysis. The interferon levels (pg) were normalized to per mg of total protein after total protein concentration for each sample was determined by BCA assay. Circle, IFN-α level within the lung in individual mice; column, mean; red bar, SEM; number, P value between specific groups when difference reached statistical significance. Other details were described in FIGS. 1A and 1G.

FIG. 2B shows that i.n. instillation of zIFV-PR8 did not appreciably induce production of IFN-β within the lung; however, the level of IFN-β in MHV1-infected and zIFV-PR8-immunized animals tended to be higher than that in MHV1-infected animals without zIFV-PR8 immunization, as well as that in zIFV-PR8-immunized animals without MHV1 infection, even though the differences did not reach statistical significance. Other details were described in FIG. 2A.

FIG. 2C shows that i.n. instillation of zIFV-PR8 did not appreciably induce production of IFN-γ within the lung; however, the level of IFN-γ in MHV1-infected and zIFV-PR8-immunized animals tended to be higher than that in MHV1-infected animals without zIFV-PR8 immunization, as well as that in zIFV-PR8-immunized animals without MHV1 infection, even though the differences did not reach statistical significance. Other details were described in FIG. 2A.

FIG. 2D shows that the mouse coronavirus MHV1 titers in lungs could be suppressed by prior exposure to zIFV-PR8. The harnessed MHV1 stayed at low levels in zIFV-PR8-immunized animals without lethal effect and were eradicated 27 days post-MHV1 challenge. Circle, MHV1 titer (TCID₅₀ per gram of lung) in each mouse; column, geometric mean; red bar, geometric SD; MHV1-4d group, MHV1 titers in lungs harvested 4 days post-MHV1 challenge; MHV1-5d group, MHV1 titers in lungs harvested 5 days post-MHV1 challenge; MHV1-6d group, MHV1 titers in lungs harvested 6 days post-MHV1 challenge; zPR8-4d−MHV1-4d group, MHV titers in lungs harvested 8 days post-zIFV-PR8 immunization and 4 days post-MHV1 challenge; zPR8-8d−MHV1-4d group, MHV titers in lungs harvested 12 days post-zIFV-PR8 immunization and 4 days post-MHV1 challenge; zPR8-12d−MHV1-4d group, MHV titers in lungs harvested 16 days post-zIFV-PR8 immunization and 4 days post-MHV1 challenge; zPR8-8d−MHV1-27d group, MHV titers in lungs harvested 35 days post-zIFV-PR8 immunization and 27 days post-MHV1 challenge; zPR8-12d group, MHV titers in lungs harvested 12 days post-zIFV-PR8 immunization. Mice were challenged by i.n. instillation of 1.6×10⁴ TCID₅₀ of MHV1. Circle, MHV1 titer (TCID₅₀ per gram of lung tissue) from each animal; column, geometric mean; red bar, geometric SD; number, selected P value between critical groups; statistical analysis was performed using GraphPad Prism software; one-way ANOVA with Tukey's multiple comparison post-tests were performed for comparing viral titers in lungs; statistical significance was set at P<0.05. Other details were described in FIG. 1A legend.

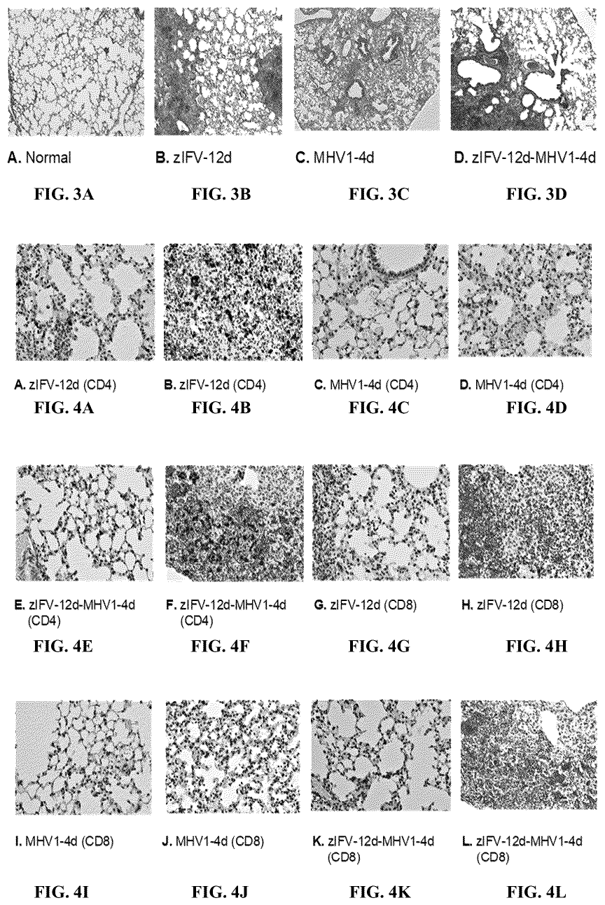

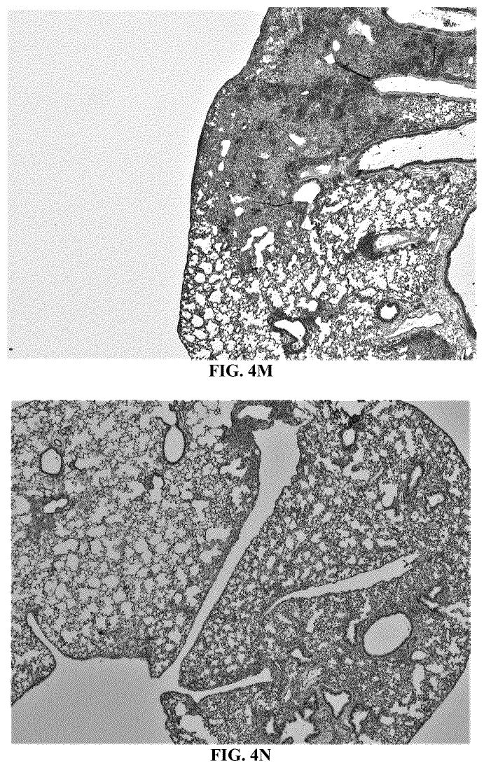

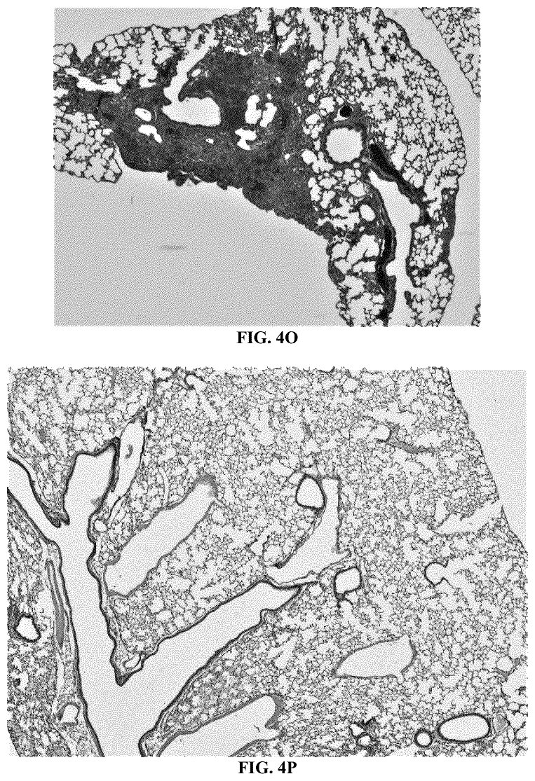

Tug of war between coronavirus-induced lymphopenia and zIFV-induced lymphocytosis. It is demonstrated that zIFV-PR8 and MHV1 induced distinct lung histopathologies in A/J mice after i.n. instillation. As shown in FIGS. 3A to 3D, zIFV-PR8 induced formation of discrete foci with densely packed cells in lungs (FIG. 3B) which were not associated with any adverse effects (FIGS. 1A to 1G). In contrast, MHV1 recruited mononuclear cell infiltrates into alveolar spaces with nodular formation within inflamed regions in lungs (FIG. 3C) which were associated with lethal effects (FIGS. 1A to 1G). When zIFV-PR8 was inoculated by i.n. instillation followed by MHV1, the pair induced foci formation with lessened inflammation in the alveolar spaces (FIG. 3D). Alveolar hemorrhage with red blood cells were found within the alveolar spaces after zIFV-PR8 and/or MHV1 were administrated by i.n. instillation (FIG. 3B-FIG. 3D). Evidence suggested that some types of lung inflammation could contribute to lethality (FIG. 3C), whereas others may be harmless (FIG. 3B) or even protective (FIG. 3D). Further investigation with immunohistochemistry (IHC) revealed that there was a tug of war between zIFV-PR8 and MHV1 in controlling the numbers of CD4⁺ and CD8⁺ T cells in lungs. As shown in FIGS. 4A to 4L, large numbers of CD4⁺ as well as CD8⁺ T cells were found within the alveolar spaces (FIG. 4A, FIG. 4G) and foci (FIG. 4B, FIG. 4H), revealed as dark brown cells in lungs harvested from A/J mice 12 days after i.n. instillation of zIFV-PR8. By contrast, few, if any, CD4⁺ and CD8⁺ T cells were found within the alveolar spaces (FIG. 4C, FIG. 4I) and inflamed regions (FIG. 4D, FIG. 4J) in lungs harvested from animals 4 days after i.n. instillation of MHV1. CD4⁺ and CD8⁺ T cells nearly vanished within alveolar spaces (FIG. 4E, FIG. 4K) whereas they were still found within foci (FIG. 4F, FIG. 4L) in lungs 4 days after challenging zIFV-PR8-immunized animals with MHV1. Results suggested that zIFV-PR8 could recruit large numbers of CD4⁺ and CD8⁺ T cells into lungs whereas MHV1 depleted CD4⁺ and CD8⁺ T cells in lungs. When MHV1 was inoculated into zIFV-PR8-immunized animals, a tug of war over the numbers of CD4⁺ and CD8⁺ T cells in lungs occurred. CD4⁺ and CD8⁺ T cells are conventionally perceived as drivers for eliciting adaptive immune responses.

FIGS. 3A to 3D show the lung histopathology induced by i.n. instillation of zIFV and/or MHV1. (FIG. 3A) Lung resected from an untreated normal A/J mouse. (FIG. 3B) Lung resected from an A/J mouse 12 days after i.n. instillation of zIFV-PR8 (800 TCID₅₀ of PR8 mixed with 500 μg of zanamivir). (FIG. 3C) Lung resected from an A/J mouse 4 days after i.n. instillation of MHV1 (2×10⁴ TCID₅₀). (FIG. 3D) Lung resected from an A/J mouse with zIFV-PR8 inoculated by i.n. instillation on Day 0, followed by i.n. instillation of MHV1 on Day 12, with the lung resected on Day 16. Mouse lungs were fixed by perfusing 10% buffered formalin in situ. Paraffin-embedded tissues were cut into 5-μm-thick slices followed by staining sections with hematoxylin and eosin. Each section is a representative of 2-3 mice. Lung sections were examined on an all-in-one microscope Keyence BZ-X800 series. Magnification X100.

FIGS. 4A to 4L show the tug of war between zIFV-PR8 and MHV1 in controlling the numbers of CD4⁺ and CD8⁺ T cells in the lung. (FIG. 4A) CD4⁺ T cells within alveolar spaces in a lung resected from an A/J mouse 12 days after i.n. instillation of zIFV-PR8 (800 TCID₅₀ of PR8 mixed with 500 μg of zanamivir). (FIG. 4B) CD4⁺ T cells within foci in the same lung as shown in (FIG. 4A). (FIG. 4C) CD4⁺ T cells within alveolar spaces in a lung resected from an A/J mouse 4 days after i.n. instillation of MHV1 (2×10⁴ TCID₅₀). (FIG. 4D) CD4⁺ T cells within inflamed region in the same lung as shown in (FIG. 4C). (FIG. 4E) CD4⁺ T cells within alveolar spaces in a lung resected from an A/J mouse with zIFV-PR8 administered by i.n. instillation on Day 0, followed by i.n. instillation of MHV1 on Day 12, with the lung resected for IHC analysis on Day 16. (FIG. 4F) CD4⁺ T cells within foci in the same lung as shown in (FIG. 4E). (FIG. 4G) CD8⁺ T cells within alveolar spaces in the same lung as shown in (FIG. 4A). (FIG. 4H) CD8⁺ T cells within foci in the same lung as shown in (FIG. 4A). (FIG. 4I) CD8⁺ T cells within alveolar spaces in the same lung as shown in (FIG. 4C). (FIG. 4J) CD8⁺ T cells within inflamed region in the same lung as shown in (FIG. 4C). (FIG. 4K) CD8⁺ T cells within alveolar spaces in the same lung as shown in (FIG. 4E). (FIG. 4L) CD8⁺ T cells within foci in the same lung as shown in (FIG. 4E). Five-μm-thick sections were cut from paraffin-embedded mouse lungs. Deparaffinization was carried out in 100% xylene. Sections were blocked followed by incubation with HRP-conjugated anti-mouse CD4 antibody (Abcam) and HRP-conjugated anti-mouse CD8 antibody (Cell Signaling), respectively. The bound peroxidase was visualized by incubating the sections with ImmPACT DAB Peroxidase (HRP) Substrate (Vector Laboratories). The slides were counterstained with modified Mayer's Hematoxylin (StatLab). Each section is a representative of 2-3 mice. Lung sections were examined on an all-in-one microscope Keyence BZ-X800 series. CD4⁺ and CD8⁺ T cells were revealed as dark brown cells. Magnification X400. FIG. 4M shows that i.n. instillation of zIFV-PR8 into A/J mice recruited a large number of CD3⁺ T cells (brown cells revealed during immunohistochemical analysis) into the lung. These T cells were found in both the alveolar spaces and foci. Magnification, 40×. FIG. 4N shows that very few CD3⁺ T cells were found in lungs after i.n. instillation of MHV1 into A/J mice. In addition, no foci were found either. Magnification, 40×. FIG. 4O shows that mice immunized by i.n. instillation of zIFV-PR8 followed by i.n. instillation of MHV1 12 days later, CD3⁺ T cells were still abundant within foci; however, these T cells were largely absent in alveolar spaces. Magnification, 40×. FIG. 4P shows that naïve A/J mice had a low level of CD3⁺ T cells within lungs prior to immunization with zIFV-PR8. Magnification, 40×.

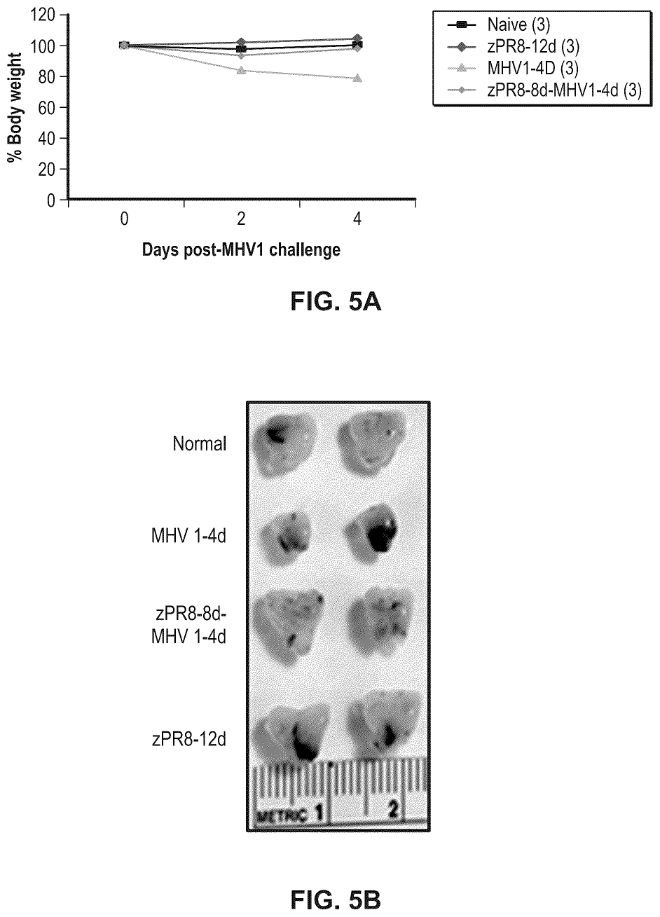

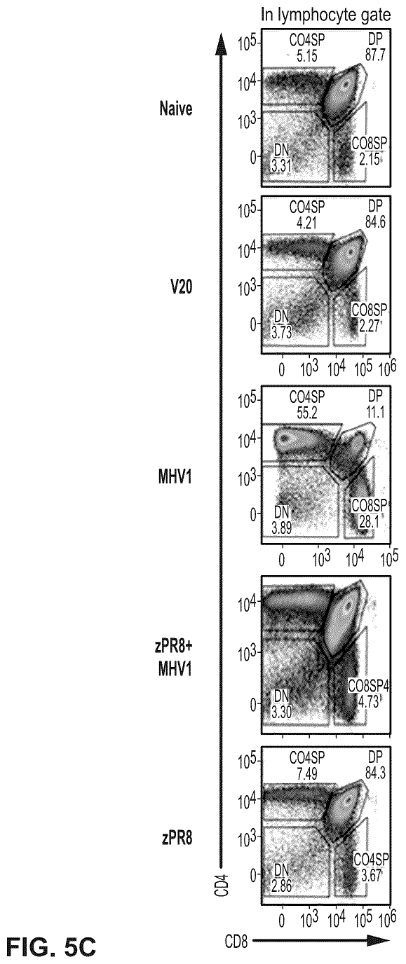

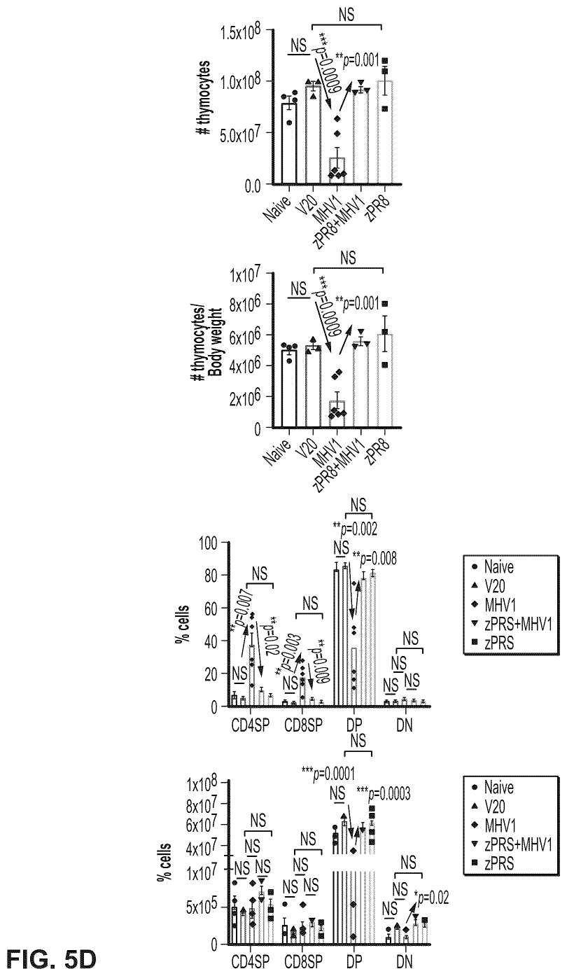

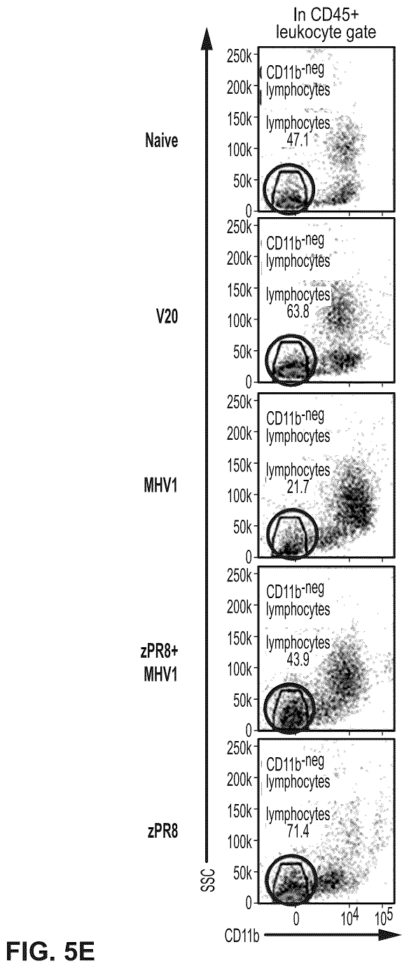

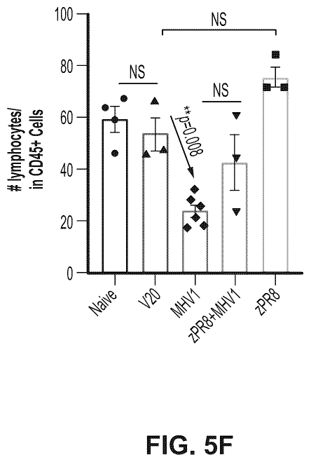

Subsequent analyses of whole blood and thymus glands with flowcytometry revealed that i.n. instillation of zIFV-PR8 could induce lymphocytosis whereas i.n. instillation of MHV1 induced lymphopenia in conjunction with thymic atrophy, and there was clearly a tug of war between lymphocytosis and lymphopenia which could explain why zIFV-PR8 recruited so many CD4⁺ and CD8⁺ T cells into the lung, MHV1 depleted nearly all of the CD4⁺ and CD8⁺ T cells in the lung, and zIFV-PR8 in conjunction with MHV1 maintained moderate numbers of CD4⁺ and CD8⁺ T cells in the lung (FIGS. 4A to 4L). As shown in FIGS. 5A to 5F, A/J mice inoculated with MHV1 quickly lost body weights whereas their counterparts inoculated with zIFV-PR8 or zIFV-PR8+MHV1 had normal body weights (FIG. 5A). MHV1 induced thymic atrophy 4 days after i.n. instillation whereas zIFV-PR8 blocked MHV1-induced thymic atrophy from occurring (FIG. 5B). Notably, CD4⁺CD8⁺ double-positive (DP) T cells plunged to nearly oblivion within thymus glands 4 days after i.n. instillation of MHV1 whereas zIFV-PR8 significantly mitigated MHV1's lethal effects on DP T cells (FIG. 5C and FIG. 5D). The % of lymphocytes in whole blood was also greatly reduced 4 days after i.n. instillation of MHV1 whereas MHV1-induced lymphopenia was mitigated by zIFV-PR8 (FIG. 5E and FIG. 5F). zIFV-PR8 alone increased the number of DP T cells within thymus glands (FIG. 5C and FIG. 5D) as well as that of lymphocytes in whole blood even though the increases did not reach statistical significance (FIG. 5E and FIG. 5F), suggesting that zIFV-PR8 may be able to induce mild lymphocytosis which could mitigate MHV1-induced lymphopenia through a tug of war.

FIGS. 5A to 5F summarize the tug of war between zIFV-PR8-induced lymphocytosis and MHV1-induced lymphopenia in A/J mice. (FIG. 5A) Body weight loss post-MHV1 challenge. Post-MHV1 challenge body weights were presented as mean % body weight by taking the body weight of individual mice on Day 0 as 100%. Naïve, control A/J mice without treatment; zPR8-12d, zIFV-PR8 (800 TCID₅₀ of PR8 mixed with 500 μg of zanamivir) were administered into each mouse by i.n. instillation, with mice euthanized 12 days later; MHV1-4d, MHV1 (2×10⁴ TCID₅₀) were administered into each mouse by i.n. instillation, with mice euthanized 4 days later; zPR8-8d−MHV1-4d, zIFV-PR8 were administered into each mouse by i.n. instillation on Day 0, followed by i.n. instillation of MHV1 on Day 8, with mice euthanized on Day 12. Numbers in parentheses represent the number of animals in each group. Statistical analysis was performed using GraphPad Prism software. Mice in the MHV1-4d group significantly lost body weight over time when compared to their counterparts in other groups (unpaired t-test). Statistical significance was set at P<0.05. (FIG. 5B) MHV1-induced thymic atrophy. Normal, thymus glands resected from naïve A/J mice; zPR8-12, thymus glands from mice in the zPR8-12d group; MHV1-4d, thymus glands from the MHV1-4d group; zPR8-8d−MHV1-4d, thymus glands from the zPR8-8d−MHV1-4d group. (FIG. 5C) Representative flowcytometric gating of thymocytes in each group of mice. (FIG. 5D) Summarized data of total thymocyte number (topmost), thymocyte number/mouse body weight (second top), percentage of thymocyte subsets (second bottom), and number of thymocyte subsets (bottommost) in each group of mice. (FIG. 5E) Representative flowcytometric gating of lymphocytes in the peripheral blood in each group of mice. (FIG. 5F) Summarized data of percentage of lymphocytes in the peripheral blood in each group of mice. Naïve, naïve A/J mice without treatment; V20, i.n. instillation of 30 μl of V20 buffer into each mouse 4 days prior to euthanasia; MHV1, the MHV1-4d group; zPR8+MHV1, the zPR8-8d−MHV1-4d group, zPR8, the zPR8-12d group. Purified PR8 and MHV1 were both stored in V20 buffer at −80° C. Single-cell suspensions were generated from homogenized thymus glands; and were subsequently stained with APC/Cy7-conjugated anti-mouse CD4 (BioLegend, cat #100414) and PE-conjugated anti-mouse CD8 (BioLegend, cat #100708). Single-cell suspensions from whole blood were stained with PerCP/Cy5.5-conjugated anti-mouse CD45 (BioLegend, cat #103132) and Brilliant Violet-conjugated anti-mouse CD11b (BioLegend, cat #101242). Data were acquired using BD™ LSR II flowcytometer and analyzed using FlowJo™ v10 software. In panels D and F, P values between multiple groups were analyzed by one-way ANOVA with Tukey's post hoc test. Statistical significance was set at P<0.05. “NS” stands for “not significant.”

As such, zIFV activates a cascade of biological events that lead to T-cell lymphocytosis (FIGS. 5A to 5F) and prime the lung for amplification of CoV-induced IFN responses (FIGS. 2A to 2C). The present invention can be used to mitigate lymphopenia induced by viral infections, bacterial infections, radiation, and senescence, as well as enhance IFN-mediated antiviral responses.

The present invention is a pathogen/mutation-agnostic antiviral/vaccine potentiator/influenza vaccine combo against a myriad of known as well as unknown airborne viruses. Specifically, it is demonstrated herein that: a) zIFV can be generated easily and quickly by mixing a live IFV with a neuraminidase inhibitor, zanamivir, in vitro; b) zIFV confers rapid protection of mice against CoV as a pathogen-agnostic antiviral; c) zIFV allows virulent CoV to linger harmlessly in hosts for a few days as an infection-dependent vaccine potentiator; d) zIFV alone is a novel mutation-agnostic influenza vaccine which can be generated rapidly; e) CoV and zIFV induce different patterns of lung inflammation, one is associated with lethality whereas another is associated with protection; f) CoV induces lymphopenia and thymic atrophy whereas zIFV mitigates CoV' s adverse effects by inducing lymphocytosis and protecting the thymus gland; and g) zIFV does not induce appreciable production of IFN-α, -β, and -γ in the lung, however, it amplifies CoV-induced IFN responses in the lung.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

### REFERENCES

Andrews, N., Tessier, E., Stowe, J., Gower, C., Kirsebom, F., Simmons, R., Gallagher, E., Thelwall, S., Groves, N., Dabrera, G., et al. (2022). Duration of protection against mild and severe disease by Covid-19 vaccines. N Engl J Med 386, 340-350.

Bernal, A. J., Gomes da Silva, M. M., Musungaie, D. B., Kovalchuk, E., Gonzalez, A., Delos Reyes, V., Martín-Quirós, A., Caraco, Y., Williams-Diaz, A., Brown, M. L., et al. (2022). Molnupiravir for oral treatment of Covid-19 in nonhospitalized patients. N Engl J Med 386, 509-520.

Bernal, J. L., Andrews, N., Gower, C., Gallagher, E., Simmons, R., Thelwall, S., Stowe, J., Tessier, E., Groves, N., Dabrera, G., et al. (2021). Effectiveness of Covid-19 Vaccines against the B.1.617.2 (Delta) Variant. N Engl J Med 385, 585-594.

Evans, R. K., Nawrocki, D. K., Isopi, L. A., Williams, D. M., Casimiro, D. R., Chin, S., Chen, M., Thu, D.-M., Shiver, J. W., and Volkin, D. B. (2004). Development of stable liquid formulations for adenovirus-based vaccines. J Pharm Sci 93, 2458-2475.

Gubareva, L. V., Kaiser, L., and Hayden, F. G. (2000). Influenza virus neuraminidase inhibitors. Lancet 355, 827-835.

Hoelscher, M. A., Garg, S., Bangari, D. S., Belser, J. A., Lu, X., Stephenson, I., Bright, R. A., Katz, J. M., Mittal, S. K., and Sambhara, S. (2006). Development of adenoviral-vector-based pandemic influenza vaccine against antigenically distinct human H5N1 strains in mice. Lancet 367, 475-481.

Kozlov, M. (2022). Why scientists are racing to develop more COVID antivirals. Nature 601, 496.

Lund, F. E., and Randall, T. D. (2021). Scent of a vaccine. Science 373, 397-399.

Parums, D. V. (2022). Editorial: Current status of oral antiviral drug treatments for SARS-CoV-2 infection in non-hospitalized patients. Med Sci Monit 28, e935952.

Poland, G. A., Jacobson, R. M., and Ovsyannikova, I. G. (2009). Influenza virus resistance to antiviral agents: a plea for rational use. Clin Infect Dis 48, 1254-1256.

Reed, L. J., and Muench, H. (1938). A simple method of estimation of 50% end points. Am J Hyg 27, 493-497.

Rubin, R. (2022). From positive to negative to positive again-the mystery of why COVID-19 rebounds in some patients who take paxlovid. JAMA 327, 2380-2382.

Sanyal, M., Holmes, T. H., Maecker, H. T., Albrecht, R. A., Dekker, C. L., He, X.-S., and Greenberg, H. B. (2019). Diminished B-cell response after repeat influenza vaccination. J Infect Dis 219, 1586-1595.

Service, R. F. (2022). Bad news for Paxlovid? Resistance may be coming. Science 377, 138-139.

Servick, K., Couzin-Frankel, J., and Matacic, C. (2021). Medicine's longest year. Science 371, 1195-1199.

Shi, Z., Zeng, M., Yang, G., Siegel, F., Cain, L. J., Van Kampen, K. R., Elmets, C. A., and Tang, D. C. (2001). Protection against tetanus by needle-free inoculation of adenovirus-vectored nasal and epicutaneous vaccines. J Virol 75, 11474-11482.

Sidik, S. M. (2022). New COVID drugs face delays as trials grow more difficult. Nature 606, 637.

Swaminathan, L., Kaatz, S., Chubb, H., Tae, K., Ramesh, M. S., Fadel, R., Big, C., Jones, J., Flanders, S. A., and Prescott, H. C. (2022). Impact of early corticosteroids on preventing clinical deterioration in non-critically ill patients hospitalized with COVID-19: A multi-hospital cohort study. Infect Dis Ther 11, 887-898.

Tang, D. C., Zhang, J., Toro, H., Shi, Z., and Van Kampen, K. R. (2009). Adenovirus as a carrier for the development of influenza virus-free avian influenza vaccines. Expert Rev Vaccines 8, 469-481.

Telenti, A., Arvin, A., Corey, L., Corti, D., Diamond, M. S., Garciá-Sastre, A., Garry, R. F., Holmes, E. C., Pang, P. S., and Virgin, H. W. (2021). After the pandemic: perspectives on the future trajectory of COVID-19. Nature 596, 495-504.

Tomazini, B. M., Maia, I. S., Cavalcanti, A. B., Berwanger, O., Rosa, R. G., Veiga, V. C., Avezum, A., Lopes, R. D., Bueno, F. R., Silva, M. V. A. O., et al. (2020). Effect of dexamethasone on days alive and ventilator-free in patients with moderate or severe acute respiratory distress syndrome and COVID-19: The CoDEX randomized clinical trial. JAMA 324, 1307-1316.

van Doremalen, N., Purushotham, J. N., Schulz, J. E., Holbrook, M. G., Bushmaker, T., Carmody, A., Port, J. R., Yinda, C. K., Okumura, A., Saturday, G., et al. (2021). Intranasal ChAdOx1 nCoV-19/AZD1222 vaccination reduces viral shedding after SARS-CoV-2 D614G challenge in preclinical models. Sci Transl Med 13, eabh0755.

Van Kampen, K. R., Shi, Z., Gao, P., Zhang, J., Foster, K. W., Chen, D. T., Marks, D., Elmets, C. A., and Tang, D. C. (2005). Safety and immunogenicity of adenovirus-vectored nasal and epicutaneous influenza vaccines in humans. Vaccine 23, 1029-1036.

Wang, P., Nair, M. S., Liu, L., Iketani, S., Luo, Y., Guo, Y., Wang, M., Yu, J., Zhang, B., Kwong, P. D., et al. (2021). Antibody resistance of SARS-CoV-2 variants B.1.351 and B.1.1.7. Nature 593, 130-135.

What is claimed is:

1. A method of treating a patient with an active agent capable of generating a pathogen/mutation-agnostic innate immune response, acting as an adaptive immune response potentiator, or as an influenza vaccine, the method comprising:
binding a live influenza virus with a viral neuraminidase inhibitor in vitro to form the active agent, with or without eliminating any unbound neuraminidase inhibitor; and
administering the active agent to the patient, wherein the active agent performs at least one of: eliciting a pathogen/mutation-agnostic innate immune response in the patient, potentiating an adaptive immune response with a pathogen, or generating an immune response against influenza.

2. The method of claim 1, wherein the influenza virus is a natural influenza virus, a bioengineered influenza virus, or a cold-adapted influenza virus.

3. The method of claim 1, wherein the active agent is administered without eliminating the unbound neuraminidase inhibitor.

4. The method of claim 1, further comprising eliminating any unbound neuraminidase inhibitor during purification of the active agent.

5. The method of claim 1, wherein the neuraminidase inhibitor is zanamivir, oseltamivir carboxylate, laninamivir, peramivir, or any other neuraminidase inhibitor.

6. The method of claim 1, wherein the pathogen is a virus, a bacterium, or a fungus.

7. The method of claim 1, wherein the active agent is inoculated into animals or human subjects by intranasal administration or oral inhalation.

8. The method of claim 1, further comprising administering the active agent to mitigate virus-induced lymphopenia or thymic atrophy or both by inducing lymphocytosis.

9. The method of claim 1, further comprising administering the active agent to trigger an antiviral response by amplifying virus-induced interferon production.

10. The method of claim 1, wherein the active agent triggers an innate immune response, an adaptive immune response, or both.

11. The method of claim 6, wherein the virus is at least one of: an influenza virus, a coronavirus, a respiratory syncytial virus, a rhinovirus, or a measles virus.

12. The method of claim 6, wherein the bacterium is Bacillus, Clostridium, Mycobacterium, Staphylococcus, Streptococcus, Pseudomonas, Klebsiella, Haemophilus, or Mycoplasma.

13. The method of claim 6, wherein the fungus is Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof.

14. The method of claim 1, further comprising administering the active agent to mitigate lymphopenia induced by radiation, senescence, inflammation, infection, or combinations thereof.

15. An active agent comprising a live influenza virus treated with a neuraminidase inhibitor in vitro formulated for nasal or oral administration.

16. A method of treating a patient suspected of having an infectious disease comprising:
identifying patients in need of prophylactic treatment for a pathogen, or patients that suffer from symptoms of the infectious disease; and
providing the patient with an effective amount of an active agent comprising a live influenza virus treated with a neuraminidase inhibitor in vitro, wherein the active agent triggers a protective immune response against the pathogen, wherein the immune response is selected from at least one of: an innate immune response, an adaptive immune responses, or both.

17. A method of potentiating an adaptive immune response to a pathogen comprising:
providing the patient with an effective amount of an active agent comprising a live influenza virus treated with a neuraminidase inhibitor in vitro wherein the treated pathogen lingers in an infected patient post-infection to trigger a protective immune response against the pathogen, wherein the immune response is an adaptive immune response.

18. The method of claim 17, wherein the pathogen is a virus, a bacterium, or a fungus.

19. The method of claim 18, wherein the virus is at least one of: an influenza virus, a coronavirus, a respiratory syncytial virus, a rhinovirus, or a measles virus.

20. The method of claim 18, wherein the bacterium is Bacillus, Clostridium, Mycobacterium, Staphylococcus, Streptococcus, Pseudomonas, Klebsiella, Haemophilus, or Mycoplasma.

21. The method of claim 18, wherein the fungus is Aspergillus flavus, Aspergillus fumigatus, Aspergillus nidulans, Aspergillus niger, Aspergillus terreus, Aspergillus ustus, Candida albicans, Candida alibicans, Candida glabrata, Candida lipolytica, Candida tropicalis, Candida tropicalis, Cryptococcus neoformans, Cryptococcus neoformas, Fusarium moniliforme, Geotricum candidum, Microsporum canis, Mucor circillelloides, Penicillium aurantiogriseum, Penicillium expansum, Penicillium italicum, Penicillium marneffei, Penicllium marneffeii, Rhizopus oryzaee, Sporotlirix schenckii, Syncephalastrum racemosum, Trichophyton mentagrophytes, Trichophyton rubrum, and a combination thereof.

22. A method of immunizing a patient against influenza by nasal spray or oral inhalation of a live influenza virus treated with a neuraminidase inhibitor in vitro.

23. The method of claim 22, wherein the live influenza virus is a clinically-isolated influenza virus.

24. The method of claim 22, wherein the live influenza virus is a bioengineered influenza virus.

25. The method of claim 22, wherein the live influenza virus is a cold-adapted live attenuated influenza virus (LAIV).
